(19) United States
(12) Patent Application Publication
(10) Pub. No.: US 2007/0191848 A1
(43) Pub. Date: Aug. 16, 2007

(54) Hydrogel bone plate spacer

(75) Inventors: Michael A. Wack, Warsaw, IN (US); Lisa K. Schroder, Rochester, IN (US); Hallie E. Brinkerhuff, Winona Lake, IN (US)

Correspondence Address: John F. Hoffman, Esq.;BAKER & DANIELS LLP, Suite 800, 111 East Wayne Street, Fort Wayne, IN, 46802, US

(73) Assignee: Zimmer Technology, Inc.

(21) Appl. No.: 11/344,670

(22) Filed: Feb. 1, 2006

(51) Int. Cl. A61F 2/30

(52) U.S. Cl. 606/69

(57) ABSTRACT

A hydrogel bone plate spacer is provided for placement between a bone plate and an underlying bone to form a fracture fixation construct at a bone fracture site. The spacer includes a hydrogel able to absorb fluid from the fracture site.

### FIELD OF THE INVENTION

The present invention relates to devices for treating bone fractures. More particularly, the present invention relates to bone plates.

### BACKGROUND

Bone fracture is a common orthopaedic injury. Treatment of bone fractures varies from conservative treatments such as casting to more aggressive treatments including surgical intervention. One surgical intervention commonly used is bone plating. In this procedure, the fracture is exposed by way of an incision, the fracture is reduced to return the bone pieces to their normal anatomical alignment, and a rigid bone plate is placed to bridge the fracture. Screws are installed through the plate and into the bone to achieve rigid fixation of the bone pieces relative to one another.

Bone plate fixation is based on the concept of stress shielding the fracture site to reduce motion of the fracture to promote healing. However, overly stiff fixation may lead to atrophy of the plated bone.

Furthermore, in traditional plating, the screws press the plate against the bone which results in friction between the plate and bone when the fracture site is loaded. This friction can lead to disturbance of soft tissues at the fracture site including soft tissue and periosteal stripping and damage to the periosteal vascular structures. It has been proposed to use limited contact bone plates having a shaped bone contact surface that reduces the bone contact area between the plate and bone to reduce the disturbance of soft tissues by the plate.

Finally, one challenge in the treatment of fractures by plating is bending the plate to fit the underlying bone profile. Conventional plating systems often require extensive intraoperative bending to accommodate the patient's bone geometry.

### SUMMARY

The present invention provides a bone plate spacer for placement in a gap between a bone plate and an underlying bone to form a fracture fixation construct at a bone fracture site.

In one aspect of the invention, the spacer includes a body including a hydrogel responsive to fluid from the fracture site over time to change the rigidity of the fracture fixation construct.

In another aspect of the invention, the spacer is transformable upon exposure to fluids at the fracture site from an initial relatively more flexible postoperative condition to a subsequent relatively less flexible postoperative condition.

In another aspect of the invention the spacer is able to release fluid under load to hydrodynamically lubricate the spacer/bone interface.

In another aspect of the invention, the spacer is able to swell to conform to the shape of the plate and the shape of the bone to fill a gap between the plate and bone.

In another aspect of the invention, a combination includes a bone plate spacer and a bone plate.

In another aspect of the invention, a method includes: positioning a hydrogel spacer adjacent a bone fracture; placing a bone plate over the hydrogel spacer; and inserting fasteners through the bone plate and hydrogel spacer and into the bone to form a fracture fixation construct including the plate, spacer, fasteners and bone.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a side sectional view of a bone plate and spacer according to the present invention applied to a fractured bone;

FIG. 2 is an end sectional view of the bone plate and spacer of FIG. 1;

FIG. 3 is a side sectional view of the bone plate and spacer of FIG. 1 showing expansion of the spacer;

FIG. 4 is an end sectional view of the bone plate and spacer of FIG. 1 showing expansion of the spacer;

FIG. 5 is a detail view taken from FIG. 3; and

FIG. 6 is a side sectional view of the bone plate and spacer of FIG. 1 showing the spacer as a plurality of separate spacers.

### DESCRIPTION OF THE ILLUSTRATIVE EXAMPLES

Embodiments of a hydrogel bone plate spacer include a body positionable between a bone plate and a bone to form a fracture fixation construct. The body may be initially rigid postoperatively to provide stress shielding of the fracture to promote fracture healing. The body may become less rigid over time to allow micromotion of the fracture to promote bone formation. For example, the body may include a hydrogel composition that absorbs fluid from the surgical site to transform from an initial relatively rigid postoperative condition to a subsequent relatively less rigid postoperative condition. For example, the body may include an at least partially dehydrated hydrogel in an initial relatively rigid state that softens and becomes flexible upon exposure to body fluids. In this example, the spacer may be installed tightly under the bone plate to initially stiffen the fracture fixation construct to provide stress shielding during initial healing. Softening of the hydrogel over time will result in increasing loads being transferred to the fracture site.

In another example, the spacer body may be initially flexible postoperatively to allow micromotion of the fracture to promote fracture healing. The body may stiffen over time to eventually provide more rigid fixation of the fracture. For example, the spacer may include a hydrogel composition that absorbs fluid from the surgical site to transform from an initial relatively flexible postoperative condition to a subsequent relatively less flexible postoperative condition. For example, the spacer may include an at least partially dehydrated hydrogel that swells upon contact with body fluids to tighten the fracture fixation construct.

For example, the spacer may be installed loosely under the bone plate to provide an initial relatively more flexible fracture fixation construct. As the spacer swells, it fills the gaps between the plate and bone and stiffens the construct. The spacer may include a container for the hydrogel such that the hydrogel swells against the container to stiffen the spacer. For example, the container may include a relatively inelastic, flexible covering that resists stretching but that is easily bent and able to conform to the shape of the bone and bone plate.

The container may include a film, membrane, woven construct, braided construct, and/or other suitable container. The container may be in the form of a bag, sleeve, bonded skin, and/or other suitable form. The container may include metals, polymers, and/or other suitable materials. Polymers may include polylactic acid, polyglycolic acid, polyester, polyolefin, polyimides, polyamides, polyacrylates, poly(ketones), fluropolymers, and/or other suitable polymers. For example, the container may include a relatively woven polyester sleeve sealed around the hydrogel. The container may allow fluid to diffuse into the hydrogel. The container may include a second hydrogel having a different chemical composition and/or a different level of hydration such that the spacer has an inner relatively more expandable hydrogel and an outer relatively less expandable hydrogel. As fluid penetrates to the inner hydrogel it may swell until the container is filled and resists further swelling. This swelling increases the turgidity of the spacer and thereby increases the stiffness of the fracture fixation construct.

The time for the spacer to transform may be a few hours, a few days, a few weeks, or a few months. The transformation time may be controlled by the choice of hydrogel polymers, degree of crosslinking, degree of dehydration, and the permeability of the outer surface of the spacer.

The hydrogel bone plate spacer may reduce soft tissue and bone disturbances by providing a lubricious cushion between the plate and bone. For example, the surface of the body may be soft and compliant to reduce mechanical abrasion of the tissues adjacent to the fracture site by the bone plate and/or screws. The body may provide hydrodynamic lubrication to reduce friction and thus reduce abrasion of the tissues adjacent to the fracture site. The body may include an abrasion resistant outer surface. For example, an outer container may include a flexible, relatively inelastic and abrasion resistant polymer construction.

The hydrogel bone plate spacer may be able to form itself to the shape of the plate and the underlying bone to fill differences in the shape of the plate and bone. For example, the body of the hydrogel bone plate spacer may include a hydrogel composition that swells to fill the space between the plate and bone. For example, the body may swell as it absorbs fluid from the surgical site. The act of swelling and filling the space between the plate and bone may stiffen the fracture fixation construct to provide more rigid fixation over time, cushion the bone/plate interface, and/or facilitate nutrient transport to tissues under the bone plate. For example, the spacer may be porous to facilitate the diffusion of body fluids containing nutrients through the spacer to reach the underlying bone and fracture site.

The spacer may have an elongated body corresponding generally to the shape of the bone plate. For example the spacer may be substantially the same length as the bone plate to provide a continuous spacer under the bone plate. The spacer may be smaller than the bone plate to be applied to a selected area under the bone plate. For example, the spacer may be positioned adjacent fasteners attaching the bone plate to the bone. For example, the spacer may be provided as a discrete pad that can be positioned under the plate at a screw location to form a washer under the plate. A plurality of spacers may be positioned under the bone plate to provide spacing a multiple selected location. The spacer may include one or more preformed holes for allowing fasteners to pass through the spacer. Alternatively, the spacer may be solid and a fastener may be driven through the spacer forming its own passageway as it is driven.

The hydrogel bone plate spacer body may include a hydrogel having a three dimensional network of polymer chains with water filling the void space between the macromolecules. The hydrogel may include a water soluble polymer that is crosslinked to prevent its dissolution in water. The water content of the hydrogel may range from 20-80%. The high water content of the hydrogel results in a low coefficient of friction for the bearing due to hydrodynamic lubrication. Advantageously, as loads increase on the bearing component, the friction coefficient decreases as water forced from the hydrogel forms a lubricating film. The hydrogel may include natural or synthetic polymers. Examples of natural polymers include polyhyaluronic acid, alginate, polypeptide, collagen, elastin, polylactic acid, polyglycolic acid, chitin, and/or other suitable natural polymers and combinations thereof. Examples of synthetic polymers include polyethylene oxide, polyethylene glycol, polyvinyl alcohol, polyacrylic acid, polyacrylamide, poly (N-vinyl-2-pyrrolidone), polyurethane, polyacrylonitrile, and/or other suitable synthetic polymers and combinations thereof.

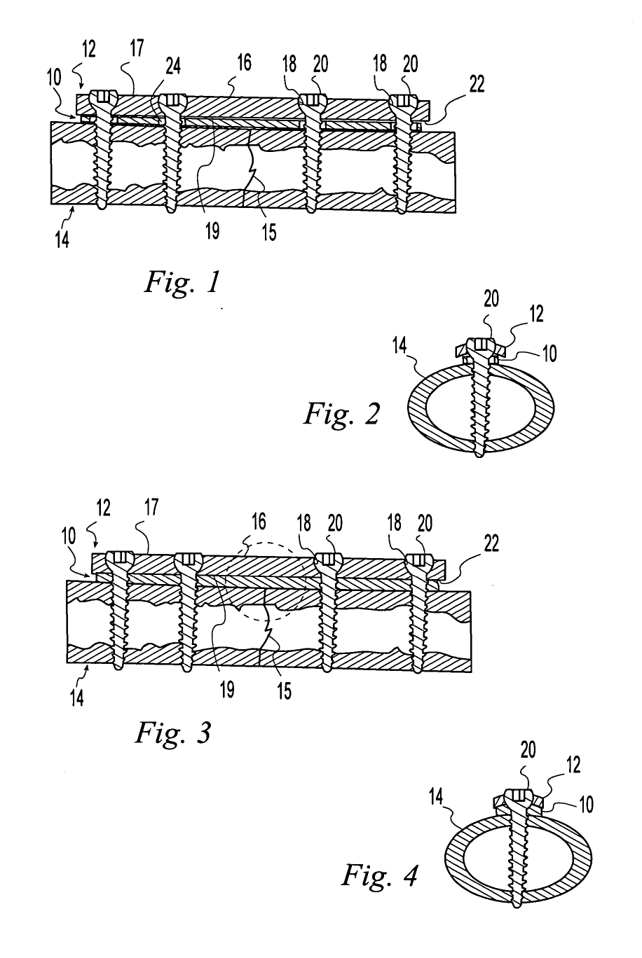

FIGS. 1-4 depict a hydrogel bone plate spacer 10 positioned between a bone plate 12 and a bone 14 including a fractured portion 15. The bone plate 12 includes an elongated body 16 having a top surface 17, a bottom surface 19, and a plurality of transverse through holes 18 extending from the top surface 17 to the bottom surface 19 for receiving fasteners such as bone screws 20. The spacer 10 has generally the same shape as the underside 22 of the bone plate 12 and includes through holes 24 for passage of the bone screws 20. The bone plate 12, spacer 10, bone 14, and screws 20 form a fracture fixation construct. The spacer 10 includes a hydrogel composition that absorbs fluids at the fracture site to change the rigidity of the fracture fixation construct over time.

In use, the fracture site is exposed by creating an incision through the overlying soft tissues. The fracture 15 is reduced by returning the bone pieces to their proper anatomic alignment and position. The spacer 10 is laid over the fracture and the plate 12 is laid over the spacer 10. Screws are inserted through the plate 12 and spacer 10 and threaded into the bone 14. The plate 12 may include elliptical holes 18 that force the screws 20 laterally as the screws 20 are tightened to apply compressive forces to the fracture 15. The plate 12 may be left loose initially to allow movement of the fracture initially. Over time, the spacer 10 absorbs fluids from the fracture site and swells to fill the space between the plate 12 and bone 14 (FIGS. 3 and 4). The swollen body stiffens the plate, spacer, bone construct and thereby provides fracture fixation that increases postoperatively from an initial relatively flexible fracture fixation to a subsequent more rigid fracture fixation. The spacer 10 also reduces soft tissue and bone disturbances by providing a compliant, nutrient transporting, and hydrodynamically lubricated bone contact surface. Finally, the spacer eliminates gaps between the plate and bone by swelling to conform to the gaps.

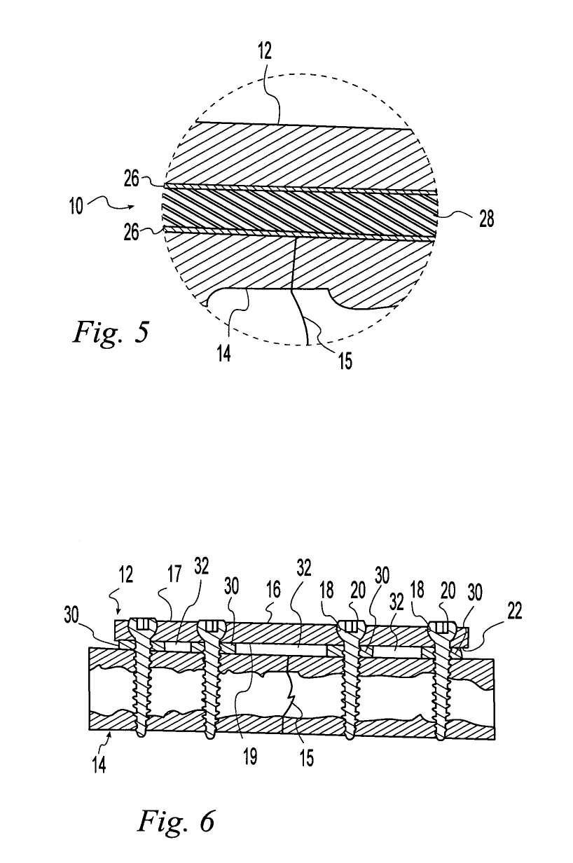

FIG. 5 is a detailed view showing an alternative construction for the spacer 10 including a flexible and relatively inelastic outer membrane 26 and a hydrogel filler 28. As the hydrogel filler 28 absorbs fluids, it expands and thus increases the turgidity of the spacer 10. Once the membrane 26 has been fully expanded, it resists further expansion of the hydrogel filler 28.

Alternatively, the spacer 10 of FIG. 1 may be initially provided as a relatively stiff spacer 10 that is placed on the bone and over which the bone plate 12 is tightly clamped to provide initially rigid fixation. As the spacer 10 absorbs fluid, it softens and becomes more flexible. Thus, over time, the fracture fixation construct transforms from initial relatively rigid fixation to subsequent relatively less rigid fixation. In this application, the hydrogel is preferably not contained or is contained in an elastic covering such that the spacer 10 does not stiffen as the hydrogel swells.

FIG. 6 illustrates an alternative configuration for the spacer 10 in which it is provided as discrete pads 30 or washer-like pieces positioned adjacent the screws 20. In this configuration, the pads 30 are responsive to fluid at the surgical site to change the stiffness of the fracture fixation construct over time similar to the earlier examples. However, the use of discrete pads 30 leaves space 32 between the plate 12 and bone 14 for nutrient ingress and where no overlying contact can abrade the bone.

Although examples of a hydrogel bone plate spacer and its use have been described and illustrated in detail, it is to be understood that the same is intended by way of illustration and example only and is not to be taken by way of limitation. Accordingly, variations in and modifications to the hydrogel bone plate spacer and its use will be apparent to those of ordinary skill in the art, and the following claims are intended to cover all such modifications and equivalents.

What is claimed is:

1. A bone plate spacer for placement between a bone plate and an underlying bone to form a fracture fixation construct at a bone fracture site, the bone plate having a top surface and a bottom surface and holes through the plate from the top surface to the bottom surface for receiving fasteners to attach the plate to the bone, the bone plate spacer comprising:
a body having a top surface, a bottom surface, and a plurality of fixation holes through the body from the top surface to the bottom surface to allow passage of the fasteners through the body, the body comprising a hydrogel responsive to fluid from the fracture site over time to change the rigidity of the fracture fixation construct.

2. The bone plate spacer of claim 1 wherein the body is transformable upon exposure to fluids at the fracture site from an initial relatively more flexible postoperative condition to a subsequent relatively less flexible postoperative condition.

3. The bone plate spacer of claim 2 wherein the body further comprises a flexible container surrounding the hydrogel, the hydrogel being expandable by absorbing fluid from the fracture site to fill the container.

4. The bone plate spacer of claim 1 wherein the body is transformable upon exposure to fluids at the fracture site from an initial relatively more rigid postoperative condition to a subsequent relatively less rigid postoperative condition.

5. The bone plate spacer of claim 4 wherein the body comprises a relatively rigid hydrogel structure that softens and becomes relatively more flexible over time upon exposure to fluid at the fracture site.

6. The bone plate spacer of claim 1 wherein the bottom surface of the body is able to release fluid under load to hydrodynamically lubricate the spacer/bone interface.

7. The bone plate spacer of claim 1 wherein the body is able to swell to conform to the shape of the plate and the shape of the bone to fill a gap between the plate and bone.

8. The bone plate spacer of claim 1 wherein the body comprises natural polymers.

9. The bone plate spacer of claim 1 wherein the body comprises at least one polymer selected from the group consisting of polyhyaluronic acid, alginate, polypeptide, collagen, elastin, polylactic acid, polyglycolic acid, and chitin.

10. The bone plate spacer of claim 1 wherein the body comprises synthetic polymers.

11. The bone plate spacer of claim 1 wherein the body comprises at least one polymer selected from the group consisting of polyethylene oxide, polyethylene glycol, polyvinyl alcohol, polyacrylic acid, polyacrylamide, poly(N-vinyl-2-pyrrolidone), polyurethane, and polyacrylonitrile

12. A combination of a bone plate spacer and a bone plate for placement over a bone at a bone fracture site to form a fracture fixation construct, the combination comprising:
a bone plate having an elongated body having a top surface, a bottom surface, and a plurality of holes extending through the body from the top surface to the bottom surface able to receive fasteners; and
a bone plate spacer, the spacer having a body with a top surface and a bottom surface and being positionable between the bone plate and the bone, the body comprising a hydrogel able to absorb fluid from the fracture site over time to change the rigidity of the fracture fixation construct.

13. The combination of claim 12 wherein the bone plate spacer further comprises a plurality of holes through the body from the top surface to the bottom surface aligned with the holes in the bone plate to allow passage of fasteners through the body

14. The combination of claim 12 wherein the body is transformable upon exposure to fluids at the fracture site from an initial relatively more flexible postoperative condition to a subsequent relatively less flexible postoperative condition.

15. The bone plate spacer of claim 14 wherein the body further comprises a flexible container surrounding the hydrogel, the hydrogel being expandable by absorbing fluid from the fracture site to fill the container.

16. The bone plate spacer of claim 12 wherein the body is transformable upon exposure to fluids at the fracture site from an initial relatively more rigid postoperative condition to a subsequent relatively less rigid postoperative condition.

17. The bone plate spacer of claim 16 wherein the body comprises a relatively rigid hydrogel structure that softens and becomes relatively more flexible over time upon exposure to fluid at the fracture site.

18. The combination of claim 12 wherein the bottom surface of the body is able to release fluid under load to hydrodynamically lubricate the spacer/bone interface.

19. The combination of claim 12 wherein the body swells to conform to the shape of the plate and the shape of the bone to fill the gap between the plate and bone.

20. The combination of claim 12 further comprising bone screws insertable through the plate and spacer to engage the bone.

21. A method of treating a bone fracture comprising:
positioning a hydrogel spacer adjacent to a bone fracture;
placing a bone plate over the hydrogel spacer; and
inserting fixation members through the bone plate and hydrogel spacer and into the bone to form a fracture fixation construct including the plate, spacer, fixation members and bone.

22. The method of claim 21 further comprising allowing the spacer to absorb fluid from the fracture site to change the rigidity of the fracture fixation construct.

23. The method of claim 22 wherein inserting the fixation members comprises attaching the bone plate and hydrogel spacer relatively loosely to the bone, the method further comprising
allowing the spacer to absorb fluid from the fracture site to swell and tighten the fracture fixation construct over time.

24. The method of claim 22 wherein inserting the fixation members comprises attaching the bone plate and hydrogel spacer relatively tightly to the bone, the method further comprising
allowing the spacer to absorb fluid from the fracture site to soften and loosen the fracture fixation construct over time.

25. The method of claim 21 further comprising allowing the spacer to absorb fluid from the fracture site to cause it to swell to conform to the shape of the plate and the shape of the bone to fill the gap between the plate and bone.
